(19) United States
(12) Patent Application Publication
(10) Pub. No.: US 2007/0172908 A1
(43) Pub. Date: Jul. 26, 2007

(54) Enteropathogenic E. Coli is a separate genus pathogenic bacteria

(76) Inventor: Yakov Yevseyevich Brilman, Voorhees, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 11/635,390

(22) Filed: Dec. 6, 2006

Related U.S. Application Data
(63) Continuation-in-part of application No. 10/972,491, filed on Sep. 23, 2003, now abandoned.

(51) Int. Cl. C12Q 1/26; C12Q 1/16

(52) U.S. Cl. 435/25; 435/35

(57) ABSTRACT

An assay is provided which recognizes the absence of dehydrogenase in all pathogenic bacteria and its presence in nonpathogenic (conditionally pathogenic) bacteria creating the opportunity to separate and establish enteropathogenic Escherichia (EPE) in a pathogenic genus bacteria; genus Brilmanella. A method for diagnosing maladies of the bowel based on the existence of the Brilmanella is also provided.

### CROSS REFERENCE TO RELATED APPLICATIONS

This application is a Continuation-in-Part application of U.S. Ser. No. 10/972,491 filed Sep. 23, 2003, the entire disclosure of which is incorporated by reference herein.

### FIELD OF THE INVENTION

The invention is related to an area of bacteriology. Specifically, the separation of enteropathogenic E. Coli in a separate genus of bacteria (Brilmanella) as established by a unique test, to differentiate pathogenic from conditionally pathogenic bacteria.

### DESCRIPTION OF RELATED ART

According to existing categorizations, bacteria in the family Enterobacteriaceae, in the genus Escherichia with type species E. Coli., are found with conditionally pathogenic and pathogenic E. Coli bacteria. It is well recognized that association of these bacteria in one genus and type, species E. Coli., is connected by similar morphological and cultural characteristics. Enterobacteries (leadership for medics), Pocrovsky V. I., Moskow, 198, p. 164-171, 307 and B. Ya. Elbert, Microbes and viruses, Minsk Belarus, 1960, p. 194-208. In the past, it has not been recognized that a separate genus, identified by the applicant as Brilmanella, exists which differs in antigenic structure, constant and typical for each strain. Applicant is unaware of any laboratory procedure that exists to separate and easily test for the Brilmanella genus so as to establish it exists as a unique genus in samples and to distinguish it from non pathogenic E. Coli. Applicant has written articles on possible tests that have now been recognized to be useful to distinguish this genus, however, there is still a need for both an assay and the recognition of this genus of E. Coli. Y. Brilman, “An additional test for identification of Enterobacteriaceae and some representatives of the genus Vibrio”, J. “Klinical laboratory diagnostics”, Moskow 1995, B 3 p. 7-8. and Y. Brilman, “The additional, dehygrogenase or oxidation-reduction test in the classification of enterobacteria”, Journal of Microbiology, Epidemiology and Immunobiology, Moskow 1998, N 4, p. 17-19.

### BRIEF SUMMARY OF THE INVENTION

The invention includes an assay which recognizes the absence of dehydrogenase in all pathogenic bacterias and its presence in nonpathogenic (conditionally pathogenic) bacteria, thereby creating the opportunity to separate and establish enteropathogenic Escherichia (EPE) as a pathogenic genus bacteria; genus Brilmanella. The assay is based on the recognition of a change in colorization of the indicators as a result of hydrogen exchange/fermentation.

The present invention also includes a pathogenic genus bacteria for diagnosis of maladies of the bowel which is a distinctive genus from the non pathogenic bacteria E. coli as recognized by color changes of the indicator.

The present invention also includes a method for diagnosing maladies of the bowel based on the existence of the Brilmanella genus.

### DETAILED DESCRIPTION OF THE INVENTION

The discovery of a differential-diagnostic test/assay, referred to herein as; oxidation—reduction test (ORT)/(OR-test), or dehydrogenase test (DGT), or as more commonly referenced, the test of the author (Brilman's test), is based on the reduction of an indicator during incubation of bacteria. This ORT, characterizing reduction of the indicator, is constantly negative and constantly positive beside conditionally pathogenic E. Coli (354B strains) in semifluid mannitol and in Kligler's medium. In the absence of this necessary laboratory test, genus Escherichia and type species E. Coli are categorized with non-pathogenic E. Coli as well as pathogenic (EPE).

As maladies exist in patients with dysfunction of the bowels, who have constantly negative ORT, the need has existed for additional special study that pertain to the genus Brilmanella. This assay, which defines the genus, is the first step in an effort to treat maladies of the bowel based on this genus. As a result of laboratory observations, (on significant material), the ORT, which recognizes differences in characteristics of separate bacteria is discovered and is the basis for a diagnositic to treat patients having maladies based on this bacteria genus.

At the core of bacteriological research on differential diagnostics of the representative family Enterobacteriaceae, is the recognition of ferments (herein defined as the simpler substances of the breakdown of an organic molecules) hydrolases, catalysis hydrolysis of the organic binding, or more simply, hydrolysis breakup of carboyhydrates. Previously, diagnostics have not taken into account the group of oxidized-reconstruction ferments, which is the basis of bacteriological breathing and fermentation. In this group, aerobic and anaerobic dehydrogenases exist. The role of these hydrogen acceptors, in the process of oxidation, exist alongside with other materials of different indicators which join with hydrogen reduction and are decolorized. Recognition of the reduction of the applicable indicator (the ferments) is the basis of the test. Further, this test has diagnostic significance with respect to the determination of other ferments, taking into account in laboratory practice: cytochrome oxidate, dehydrocarboxydase, urease, etc.

The essence of the test is performed in the following manner: bacteria are incubated on semifluid media, GISS and on Kligler's medium. Conditionally pathogenic Escherichia exhibit typical biochemical processes, under which the indicator, via fixative carbohydrase fermentation, by change of its coloration, first restores the initial color in the aerobic layer of the medium during the process of the incubation and then in the anaerobic layer (over 48-72 h. of the incubation). The oxidation-reduction process occurs through 20-24 hours of growing bacteria on semifluid mannitol and Kligler's medium, illustrating one change of the color of the indicator in its surface layer in contrast with the remaining portion of the column of the nourishing medium.

This test was discovered during a study of sick children with dysfunctional intestines by investigating 2886 strains of bacteria E. Coli without discovery of EPE. Nearly all of them (99.38%) showed that the dehydrogenase test (DGT) was positive with the exclusion of 22 strains (0.62%) which are not known serovars of EPE (or to other pathogenic Enterobacteria); wherein complete serotyping was performed including somatic (O), capsular (K), flagellar (H) antigens and also by EPE; the resulting ORT being negative. It should be recognized that if under investigation, bacteria undergoing serological typing give a positive result in reaction of agglutination with diagnosing enteropathogenic intestines bacterias “O”- and “OK”-serums which are OR-test positive in the last witnesses culture of contamination, such bacteria need dispelling for reception of the pure culture.

In regard to the patients having the excluded 22 strains of bacteria (0.62%) with negative OR-test, additional study of such bacteria is needed for a variety of pathogenic bacteria which may exist in the genus Brilmanella. Further, E. Coli having haemolytic characteristic with negative OR-test may also exist in the Brilmanella genus (these are referred to by the Applicant as Type “Y”). Moreover, other bacteria found in a patient with dysfunction of the bowels with constantly negative ORT, not related to known serovars of EPE and “E. Coil” having hemolytic characteristics with negative OR-test, may belong to variants of the bacteria Brilmanella genus.

Additional support for the effectiveness of the assay of the invention is based on experiments conducted with 112 strains of different serological types of EPE. Results of the experiments found the dehydrogenase test (DGT) was constantly negative (absence recovering the coloration of the indicator) in column semifluid mannitol and in Kligler's medium. This was also true for Salmonellas, Shigellas, Yersinias and other pathogenic bacterias, for which an absence of dehydrogenase fermentation is typical.

Research of EPE is commonly performed on serologically select growing colonies on nourishing ambience in a Petri dish. Therefore, it should be recognized that in this incubation environment (a Petri dish, with nourishing substances), enteropathogenic intestine bacteria do not differ from colonies of conditionally pathogenic E. Coli. Further, it has been ignored that entereopathogenic intestine bacteria, which are not diagnosed by any currently available set of diagnostics, have a generic OR-test that is negative. It is therefore significant to note that of the pathogenic genus of intestine bacteria under investigation, each type of colonies grew on several nourishing ambience in a Petri dish, irrespective of the results from the serological research. Fermenting incubation characteristics were analyzed after sift incubation was performed (under 37° C. 24-27 (before 48) hours ) whereupon a negative OR-test was recognized. The constant absence of dehydrogenase fermentation by pathogenic bacteria is a typical test; differentiating them by use of the assay of the present invention from conditionally pathogenic bacteria allows the selection of EPE in a separate pathogenic genus bacteria; family Enterobacteriacea—in the genus Brilmanella.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A laboratory assay for the differential diagnostics of bacteria that establishes that Enteropathogenic E. Coli is distinct from nonpathogenic E. Coli comprising the steps of:
incubating the test bacteria on an appropriate media,
viewing the color changes of the bacteria based on the oxidation process, and
assigning an indicator for recognition of the specific bacteria genus based on the color changes.

2. The assay of claim 1, wherein the assay is an oxidation reduction or dehydrogenase reaction.

3. The assay of claim 1, wherein said assay defines a pathogenic genus bacteria for diagnosis of maladies of the bowel which is a distinctive genus from the non pathogenic bacteria E. coli

4. A pathogenic genus bacteria for diagnosis of maladies of the bowel which is a distinctive genus from the non pathogenic bacteria E. coli as recognized by color changes of the indicator in an assay comprising the steps of:
incubating the test bacteria on an appropriate media,
viewing the color changes of the bacteria based on the oxidation process, and
assigning the indicator to the specific bacteria genus based on the color changes.

5. The bacteria of claim 4, wherein said bacteria has a distinctive antigenic characteristic from non pathogenic E. coli.

6. A method for diagnosing maladies of the bowel based on the existence of the Brilmanella bacteria in a patient sample comprising the steps of:
obtaining a sample from a patient,
isolating all bacteria in the sample,
incubating the isolated bacteria on an appropriate media,
viewing the color changes of the bacteria based on the oxidation process, and
assigning an indicator to the specific bacteria genus based on the color changes.
